(19) United States
(12) Patent Application Publication
(10) Pub. No.: US 2010/0297706 A1
(43) Pub. Date: Nov. 25, 2010

(54) MUTANT DNA POLYMERASES AND THEIR GENES FROM THERMOCOCCUS

(75) Inventors: Jung Hyun Lee, Gyeonggi-do (KR); Sung Gyun Kang, Gyeonggi-do (KR); Sang Jin Kim, Gyeonggi-do (KR); Kae Kyoung Kwon, Gyeonggi-do (KR); Hyun Sook Lee, Gyeonggi-do (KR); Yun Jae Kim, Gyeonggi-do (KR); Yong Gu Ryu, Seoul (KR); Seung Seob Bae, Gyeonggi-do (KR); Jae Kyu Lim, Gyeonggi-do (KR); Jung Ho Jeon, Gyeonggi-do (KR); Yo Na Cho, Gyeonggi-do (KR)

Correspondence Address: EDWARDS ANGELL PALMER & DODGE LLP, P.O. BOX 55874, BOSTON, MA, 02205, US

(73) Assignee: KOREA OCEAN RESEARCH & DEVELOPMENT INSTITUTE, Ansan, Gyeonggi-do (KR)

(21) Appl. No.: 12/525,347

(22) PCT Filed: Oct. 2, 2007
(86) PCT No.: PCT/KR2007/004827

§ 371 Date: Jul. 31, 2009

(30) Foreign Application Priority Data
Nov. 30, 2006 (KR) 10-2006-0119612

(51) Int. Cl. C12P 19/34; C12N 9/10; C07H 21/04; C12N 15/63; C12N 1/21

(52) U.S. Cl. 435/91.2; 435/193; 536/23.2; 435/91.5; 435/320.1; 435/252.3

(57) ABSTRACT

The present invention relates to mutant DNA polymerases and their genes isolated from Thermococcus sp. More specifically, the present invention relates to mutant DNA polymerases which are originally isolated from Thermococcus sp NA1. strain and produced by site-specific mutagenesis, their amino acid sequences, genes encoding said mutant DNA polymerases, their nucleic acids sequences, recombinant vectors containing said nucleic acids sequences, host cells transformed with thereof and methods for producing mutant DNA polymerase protein by using thereof. As mutant DNA polymerases according to the present invention have increased processivity by site-specific mutagenesis on exonuclease active site compared to wild type DNA polymerase, the present invention is broadly applicable for PCR in various molecular genetic technologies.

### TECHNICAL FIELD

The present invention relates to mutant DNA polymerases, their genes and their uses. More specifically, the present invention relates to mutant DNA polymerases which is originally isolated from Thermococcus sp. strain and produced by site-specific mutagenesis, their amino acid sequences, genes encoding said mutant DNA polymerases and PCR methods using thereof.

### BACKGROUND ART

The recent advance of genomic research has produced vast amounts of sequence information. With a generally applicable combination of conventional genetic engineering and genomic research techniques, the genome sequences of some hyperthermophilic microorganisms are of considerable biotechnological interest due to heat-stable enzymes, and many extremely thermostable enzymes are being developed for biotechnological purposes.

PCR, which uses the thermostable DNA polymerase, is one of the most important contributions to protein and genetic research and is currently used in a broad array of biological applications. More than 50 DNA polymerase genes have been cloned from various organisms, including thermophiles and archaeas. Recently, family B DNA polymerases from hyperthermophilic archaea, Pyrococcus and Thermococcus, have been widely used since they have higher fidelity in PCR based on their proof reading activity than Taq polymerase commonly used. However, the improvement of the high fidelity enzyme has been on demand due to lower DNA elongation ability.

The present inventors isolated a new hyperthermophilic strain from a deep-sea hydrothermal vent area at the PACMANUS field. It was identified as a member of Thermococcus based on 16S rDNA sequence analysis, and the whole genome sequencing is currently in process to search for many extremely thermostable enzymes. The analysis of the genome information displayed that the strain possessed a family B type DNA polymerase. The present inventors cloned the gene corresponding to the DNA polymerase and expressed in E. coli. In addition, the recombinant enzyme was purified and its enzymatic characteristics were examined. Therefore, the present inventors applied for a patent on the DNA polymerase having high DNA elongation and high fidelity ability (Korean Patent Application No. 2005-0094644).

Due to strong exonuclease activity and low processivity, high fidelity DNA polymerases need to improve in various applications of PCR. Accordingly, the present inventors need to develop a DNA polymerase with decreased exonuclease activity and increased processivity from the wild type TNA1_pol DNA polymerase of Korean Patent Application No. 2005-0094644.

Accordingly, as a result of continuous efforts, the present inventors have introduced site-specific mutagenesis at hyperthermophilic DNA polymerases isolated from Thermococcus sp. strain and selected mutant DNA polymerases with a changed exonuclease activity and processivity. The identified mutant DNA polymerases are useful for various PCRs. Thereby, the present invention has been accomplished.

### DISCLOSURE

### Technical Problem

It is an object of the present invention to provide processivity increased mutant DNA polymerases and their genes.

### Technical Solution

The present invention provides processivity increased mutant DNA polymerases produced by site-specific mutagenesis on exonuclease active site from the wild type TNA1_pol DNA polymerase, Korean Patent Application No. 2005-0094644, which is isolated from Thermococcus sp. strain.

Preferably, said exonuclease active site can be one or more motifs selected from the group consisting of ExoI motif, ExoII motif and ExoIII motif.

Also, the present invention provides mutant DNA polymerases produced by one or more mutagenesis simultaneously.

### DESCRIPTION OF DRAWINGS

FIG. 1 shows the results of SDS-PAGE analysis of TNA1_pol mutant proteins. The molecular mass standards (lane M) include: phosphorylase b (103 kDa), bovine serum albumin (97 kDa), ovalbumin (50 kDa), carbonic anhydrase (34.3 kDa), soybean trypsin inhibitor (28.8 kDa), and lysozyme (20.7 kDa). 1, D141A; 2, N210D; 3, D212A; 4, N213D; 5, D215A; 6, Y311F; 7, G211D; 8, N213E; 9, N213F; 10, N213A; 11, N213R; 12, F214D

FIG. 2 shows the results of comparison of processivity among wild-type and mutants. Each trace represents one lane from a sequencing gel and each peak represents a single primer extension product. This shows electropherogram traces of wild-type and mutated DNA polymerases. Enzymes are indicated at the right-top. The labels on the x-axis indicate the primer extension product length, which is determined based on size markers run on the same gel.

FIG. 3 shows the results of comparison of PCR amplification among wild-type and mutants. 1.3 unit of wild-type and mutated DNA polymerases were used to amplify 2 kb target from λ DNA. After a single 1 min denaturation step at 95° C., 30 cycles with a temperature profile of 20 sec at 95° C., various time (5, 10, 30, 60 sec) at 72° C. were performed, PCR products were analyzed by 0.8% agarose gel electrophoresis. Enzymes and elongation times in the study are indicated at the top. Lane M, DNA molecular size marker.

FIG. 4 shows a cleavage map of recombinant plamid according to the present invention.

### BEST MODE

According to a first aspect, the present invention provides a DNA polymerase comprising any one amino acids sequence selected from the group consisting of from 3 to 9 of SEQ ID NO: 1, from 3 to 9 of SEQ ID NO: 2, from 3 to 9 of SEQ ID NO: 3, from 3 to 9 of SEQ ID NO: 4, from 3 to 9 of SEQ ID NO: 5, from 3 to 9 of SEQ ID NO: 6, from 3 to 9 of SEQ ID NO: 7, from 3 to 9 of SEQ ID NO: 8, from 3 to 9 of SEQ ID NO: 9, from 3 to 9 of SEQ ID NO: 10, from 3 to 9 of SEQ ID NO: 11 and from 3 to 9 of SEQ ID NO: 12. Also, the present invention provides a nucleotide sequence encoding any one amino acids sequence selected from the group consisting of SEQ ID NO: 13 to SEQ ID NO: 24 (full-length sequences of protein).

According to a second aspect, the present invention provides a method of DNA polymerization and PCR by using the DNA polymerase.

According to a third aspect, the present invention provides an expression vector comprising a mutant DNA polymerase gene, and a method for producing a DNA polymerase using host cells transformed with said expression vector. More specifically, the present invention provides a method for producing a DNA polymerase comprising culturing cells transformed with an expression vector comprising a mutant DNA polymerase gene inducing expression of the recombinant protein according to the present invention and purifying the mutant DNA polymerase.

As used herein, the term “DNA polymerase” refers to an enzyme that synthesizes DNA in the 5′->3′ direction from deoxynucleotide triphosphate by using a complementary template DNA strand and a primer by successively adding nucleotide to a free 3′-hydroxyl group. The template strand determines the sequence of the added nucleotide by Watson-Crick base pairing.

As used herein, the term “functional equivalent” is intended to include amino acid sequence variants having amino acid substitutions in some or all of a DNA polymerase, or amino acid additions or deletions in some of the DNA polymerase. The amino acid substitutions are preferably conservative substitutions. Examples of the conservative substitutions of naturally occurring amino acids as follow; aliphatic amino acids (Gly, Ala, and Pro), hydrophobic amino acids (Ile, Leu, and Val), aromatic amino acids (Phe, Tyr, and Trp), acidic amino acids (Asp, and Glu), basic amino acids (His, Lys, Arg, Gln, and Asn), and sulfur-containing amino acids (Cys, and Met). It is preferable that the deletions of amino acids in DNA polymerase are located in a region where it is not directly involved in the activity of the DNA polymerase.

The present invention provides a DNA fragment encoding the mutant DNA polymerase. As used herein, the term “DNA fragment” includes sequence encoding the DNA polymerase, their functional equivalents and functional derivatives. Furthermore, the present invention provides various recombination vectors containing said DNA fragment, for example a plasmid, cosmid, phasimid, phase and virus. Preparation methods of said recombination vector are well known in the art.

As used herein, the term “vector” means a nucleic acid molecule that can carry another nucleic acid bound thereto. As used herein, the term “expression vector” is intended to include a plasmid, cosmid or phage, which can synthesize a protein encoded by a recombinant gene carried by said vector. A preferred vector is a vector that can self-replicate and express a nucleic acid bound thereto.

As used herein, the term “transformation” means that foreign DNA or RNA is absorbed into cells to change the genotype of the cells.

Cells suitable for transformation include prokaryotic, fungal, plant and animal cells, but are not limited thereto. Most preferably, E. coli cells are used.

Hereinafter, the present invention will be described in further detail with reference to examples. It is to be understood, however, that these examples are for illustrative purposes only and are not to be construed to limit the scope of the present invention.

### MODE FOR INVENTION

### Example 1

### Preparation and Cloning of Mutant TNA1_pol DNA Polymerase Genes

Strains and Culture Conditions

Thermococcus sp. NA1 was isolated from a deep-sea hydrothermal vent area in the East Manus Basin of the PACMANUS field (3°14′ S, 151°42′ E). YPS medium was used to culture the archaeon for DNA manipulation [Holden, J. F. et al, FEMS Microbiol Ecol. 36 (2001) 51-60]. Culture and strain maintenance were performed according to standard procedures [Robb, F. T. et al, Archaea: a laboratory manual, Cold Spring Harbor, N.Y. pp. 3-29 (1995)]. To prepare a seed culture of Thermococcus sp. NA1, YPS medium in a 25-ml serum bottle was inoculated with a single colony from a phytagel plate and cultured at 85° C. for 20 h. Seed cultures were used to inoculate 700 ml of YPS medium in an anaerobic jar and cultured at 85° C. for 20 h. E. coli strain DH5α was used for plasmid propagation and nucleotide sequencing. E. coli strain BL21-CodonPlus(DE3)-RIL cells (Stratagene, LaJolla, Calif.) and the plasmid pET-24a(+) (Novagen, Madison, Wis.) were used for gene expression. E. coli strains were cultivated in Luria-Bertani medium with 50 μg/ml kanamycin at 37° C.

DNA Manipulation and Sequencing

DNA manipulations were performed using standard procedures, as described by Sambrook and Russell [Sambrook, J. & Russell, D. W., Molecular cloning: a laboratory manual, 3^{rd} ed., Cold Spring Harbor, N.Y. (2001)]. Genomic DNA of Thermococcus sp. NA1 was isolated using a standard procedure [Robb, F. T. et al, Archaea: a laboratory manual, Cold Spring Harbor, N.Y. pp. 3-29 (1995)]. Restriction enzymes and other modifying enzymes were purchased from Promega (Madison, Wis.). Small-scale preparation of plasmid DNA from E. coli cells was performed using a plasmid mini-prep kit (Qiagen, Hilden, Germany). DNA sequencing was performed using an ABI3100 automated sequencer, using a BigDye terminator kit (PE Applied Biosystems, Foster City, Calif.).

Construction of Mutated DNA Polymerase Genes

All site-specific mutagenesis were carried out using Quick change Kit (Stratagene, La Jolla, Calif.) according to manufacturer's instruction. TNA1_pol gene was subcloned into NdeI/XhoI site of pET-24a(+) vector, and then the resulting plasmid was used as template for the mutation. Primers for the mutation were listed in Table 1.

TABLE 1
Primer sequences for mutant construction in
the present invention

| | Position of | |
|---|---|---|
| Name of | amino acid | |
| mutants | substitution | Mutation primer sequence (5′-3′) |
| Exo I motif | | |
| D141A | Asp→Ala | ATGCTCGCCTTTGCCATCGAGACGCTCTACCACGAGGGC |
| | | (SEQ ID NO: 25) |
| Exo II motifs | | |
| N210D | Asn→Asp | CTCATTACCTACGACGGCGACAACTTTGACTTTGCTTAC |
| | | (SEQ ID NO: 26) |
| G211D | Gly→Asp | ATTACCTACAACGACGACAACTTTGACTTTGCTTACCTC |
| | | (SEQ ID NO: 27) |
| D212A | Asp→Ala | ACCTACAACGGCGCCAACTTTGACTTTGCTTACCTC |
| | | (SEQ ID NO: 28) |
| N213A | Asn→Ala | TACAACGGCGACGCCTTTGACTTTGCTTACCTC |
| | | (SEQ ID NO: 29) |
| N213D | Asn→Asp | TACAACGGCGACGACTTTGACTTTGCTTACCTC |
| | | (SEQ ID NO: 30) |
| N213E | Asn→Glu | TACAACGGCGACGAGTTTGACTTTGCTTACCTC |
| | | (SEQ ID NO: 31) |
| N213F | Asn→Phe | TACAACGGCGACTTCTTTGACTTTGCTTACCTC |
| | | (SEQ ID NO: 32) |
| N213R | Asn→Arg | TACAACGGCGACCGCTTTGACTTTGCTTACCTC |
| | | (SEQ ID NO: 33) |
| F214D | Phe→Asp | AACGGCGACAACGACGACTTTGCTTACCTCAAAAAACG |
| | | (SEQ ID NO: 34) |
| D215A | Asp→Ala | GGCGACAACTTTGCCTTTGCTTACCTCAAAAAACGTTGC |
| | | (SEQ ID NO: 35) |
| Exo III motif | | |
| Y311F | Tyr→Phe | CGCGTTGCGCGCTTCTCTATGGAAGATGCAAAGGCAACC |
| | | (SEQ ID NO: 36) |

Results

We isolated a hyperthermophilic archaeon, Thermococcus sp. NA1, cloned a family B type DNA polymerase (Korean Patent Application no. 2005-0094644). The gene contained a putative 3′-5′ exonuclease domain and an α-like DNA polymerase domain, consisting of 2322 by (Korean Patent Application no. 2005-0094644). In a pair-wise alignment with other DNA polymerases, the deduced amino acid sequence of TNA1_pol showed 91.0% identity with KOD DNA polymerase (accession no. D29671), 82.0% identity with Deep vent DNA polymerase, and 79.0% identity with Pfu DNA polymerase (accession no. D12983). Family B type DNA polymerases from two hyperthermophilic archaea, Pyrococcus and Thermococcus, have been getting popular in PCR because they offer higher fidelity than the Taq DNA polymerase [Lundberg, K. S. et al, Gene, 108 (1991) 1-6, Mattila, P. et al, Nucl. Acids Res. 19 (1991) 4967-4973, Kong, H. et al, J. Biol. Chem. 268 (1993) 1965-1975, Southworth, M. W. et al, Proc. Natl. Acad. Sci. USA, 93 (1996) 5281-5285, Takagi, M. et al, Appl. Environ. Microbiol. 63 (1997) 4504-4510]; however, there is demand for the improvement of high-fidelity enzymes because of their low elongation rates [Barnes, W. M. Proc. Natl. Acad. Sci. USA 91 (1994) 2216-2220]. TNA1_pol is a family B type DNA polymerase, containing 3′→5′ exonuclease domains (ExoI, ExoII, and ExoIII). To improve the processivity, it was postulated that mutations at the exonuclease domains could affect the processivity. To check the possibility, several mutants at ExoI, ExoII, ExoIII were introduced as described in material and method using the primers (Table 1), and the mutant constructs were expressed in E. coli.

### Example 2

### Characterization of TNA1_pol Mutants

Expression and Purification of the Wild-Type and Mutated TNA1 DNA Polymerases

The DNA fragments including the site-directed mutation were transformed into E. coli BL21-Roseta strain. Overexpression of the mutated genes were induced by addition of isopropyl-β-D-thiogalactopyranoside (IPTG) at the mid-exponential growth phase, follow by 3-h incubation at 37° C. The cells were harvested by centrifugation (6000×g at 4° for 20 min) and resuspended in 50 mM Tris-HCl buffer (pH 8.0) containing 0.1 M KCl and 10% glycerol. The cells were disrupted by sonication; and after centrifuged (20,000×g at 4° C. for 30 min), a crude enzyme sample was prepared by heat treatment at 80° for 20 min. The resulting supernatant was applied to a column of TALON™ metal affinity resin (BD Biosciences) and washed with 5 mM imidazole (Sigma) in 50 mM Tris-HCl buffer (pH 8.0) containing 0.1 M KCl and 10% glycerol; and enzyme was eluted in the same buffer with 300 mM imidazole. The pooled fractions were dialyzed into storage buffer containing 50 mM Tris-HCl (pH 7.5), 1 mM DTT, 1 mM EDTA, and 10% glycerol.

The protein concentration was determined by Bradford assay, and protein purity was examined using sodium dodecyl sulfate-polyacrylamide gel electrophoresis (SDS-PAGE), using standard procedures.

Exonuclease Activity Assay

Exonuclease activity was measured using 3′ end-labeled DNA and 5′ end-labeled DNA as substrates. In brief, pBluescript SK plasmid, linearized by NotI, was filled in by Klenow fragment in the presence of [α-³²P]dCTP, and a 2-kb PCR product was phosphorylated by T4 polynucleotide kinase in the presence of [γ-³²P]ATP. After labeling, the DNA substrates were purified by ethanol precipitation and incubated with the enzyme in a 25 ul reaction mixture consisting of 20 mM Tris-HCl (pH 7.5), 10 mM MgCl₂, 1 mM 2-mercaptoethanol, 20 mM (NH₄)₂SO₄, and 0.01% bovine serum albumin at 75° C. for 10 min in the presence or absence of dNTPs. The reaction was precipitated by adding 1 ml of 5% trichloroacetic acid in the presence of BSA as a carrier. After centrifugation, the supernatant was withdrawn and its radioactivity was counted using a Beckman LS6500 scintillation counter.

Fidelity Assay

The error rate of TNA1_pol during PCR was determined by direct sequencing. A 2-kb target from λ DNA was amplified using 1.25 unit (U) of TNA1 DNA polymerases. The PCR products were cloned into pCRII-TOPO (Invitrogen) and transformed into E. coli DH5α. Fifty clones from each reaction were randomly selected, and the fragments of interest were sequenced. The error rate was calculated as the ratio of the number of error to the total nucleotides read.

Assay for Processivity

Processivity of mutated DNA polymerase was measured by means of the method previously reported. A 5′ Hex-labeled M13-primer (400 fmol) was added to M13mp18 ssDNA (200 fmol) in a reaction mixture containing 20 mM Tris-HCl (pH 8.5), 1 mM MgCl₂, 60 mM KCl, 30 mM (NH₄)₂SO₄, and 0.2 mM dNTPs. The mixture was preheated at 95° C. for 1 min, and incubated at 62° C. for 1 min using T1 thermocycler (Biometra). Finally, DNA polymerases were added to the mixture, which was incubated at 75° C. for 10 sec. The resulting DNA fragments were analyzed using an ABI3100 automated sequencer.

Results

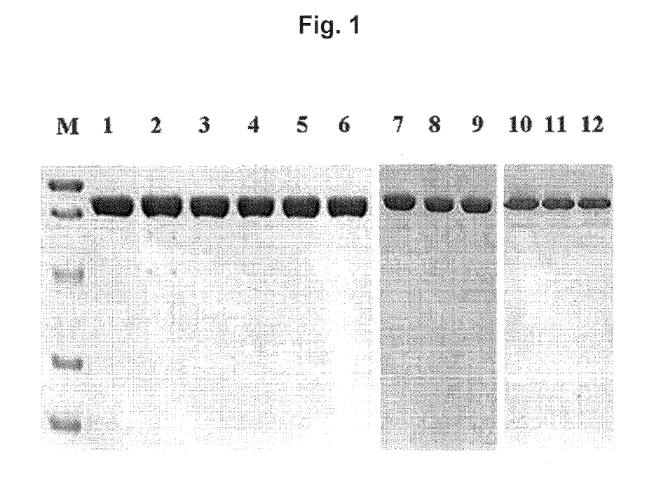

The recombinant mutant proteins of TNA1_pol were soluble and purified using TALON™ metal affinity chromatography. SDS-PAGE revealed that mutant proteins were similar to wild type TNA1_pol, showing major protein band with a molecular mass of 90 kDa (FIG. 1). The purified proteins remained soluble after repeated freezing and thawing cycles.

As shown in Table 2, the mutations at the critical residues of ExoI, ExoII, and ExoIII such as D141A, N210D, D215A, Y311F significantly abolished the exonuclease activities of TNA1_pol. However, the other mutant proteins at ExoII domain except N210 and D215 were largely showing similar exonuclease activity to wild type protein.

TABLE 2
Comparison of 3′-5′ exonuclease activity and error
rate among wild type and mutated DNA polymerases

| DNA | Relative exonuclease | |
|---|---|---|
| polymerases | activity [%] | Error rate^{a} |
| Wild-type | 100 | 2.2 × 10⁻⁴ |
| D141A | 28 | ND^{b} |
| N210D | 42 | ND |
| G211D | 30 | ND |
| D212A | 98 | ND |
| N213A | 96 | ND |
| N213D | 97 | 3.2 × 10⁻⁴ |
| N213E | 100 | ND |
| N213F | 101 | ND |
| N213R | 101 | ND |
| F214D | 27 | ND |
| D215A | 29 | ND |
| Y311F | 43 | ND |
| rTaq | ND | 1.6 × 10⁻³ |

^{a}Error rate was calculated as erroneous nt/total nt
^{b}ND indicated ‘not determined’.

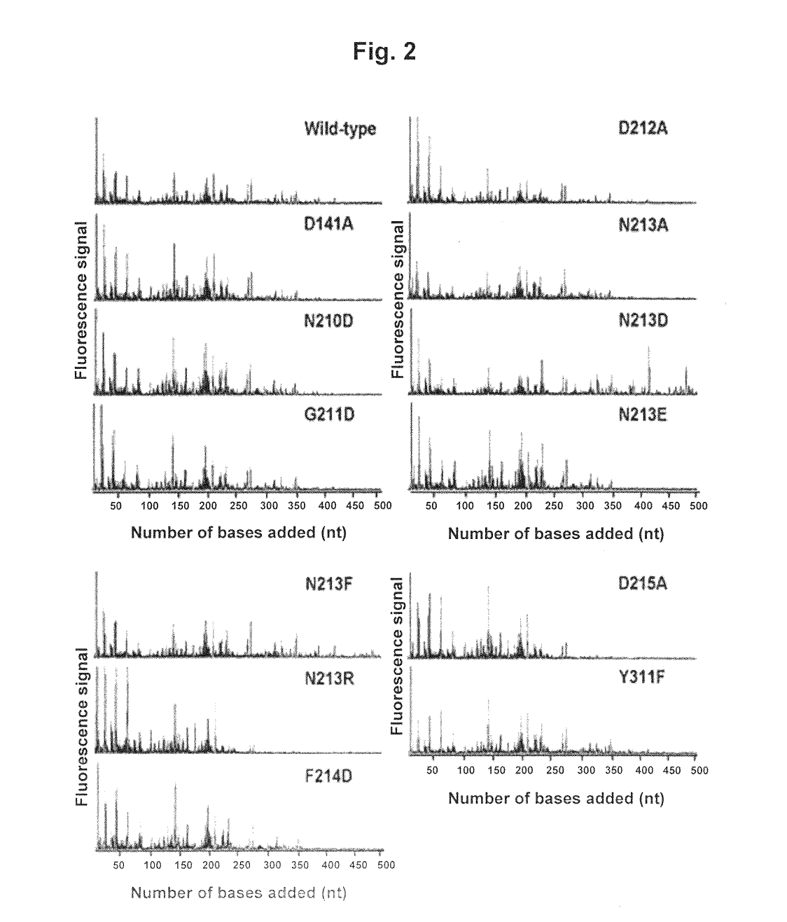

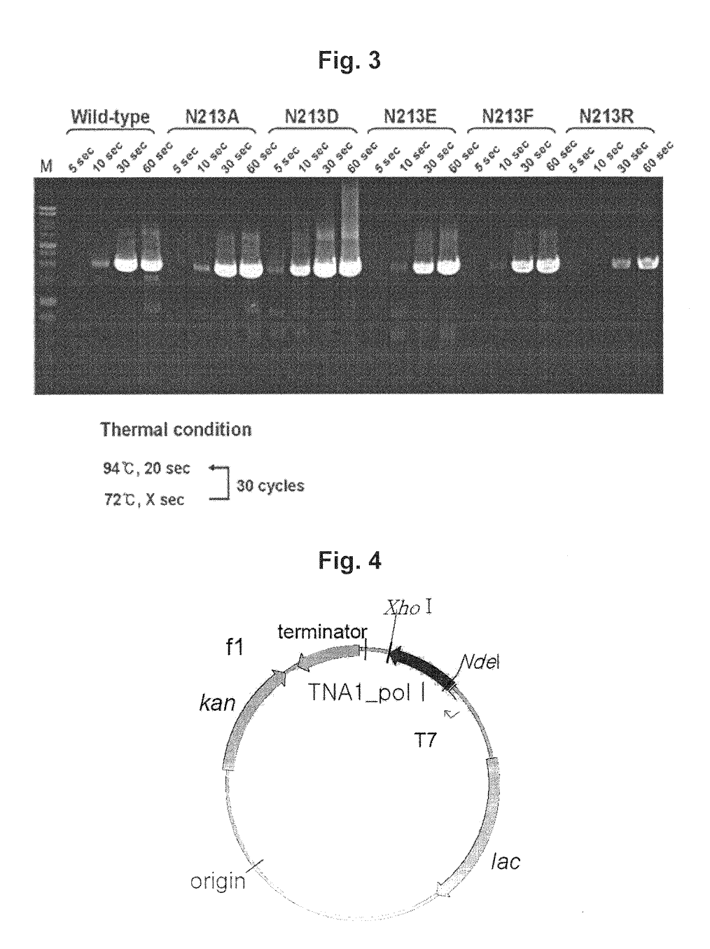

Most of mutant proteins showed similar fluoregenic profiles in the assay of processivity determination, indicating that the processivity of the mutant proteins was not changed significantly (FIG. 2). However, the fluoregenic profile of N213D was significantly changed, compared to that of wild type. The processivity of N213D was determined to be 400 nt, which is three fold higher than that of wild type. The increased processivity of N213D could be confirmed by PCR amplification experiment with extension time varied (FIG. 3). Most of mutant proteins including wild type protein yielded target bands at 30 sec but N213D mutant protein could yield the target band within 10 sec. On the contrary, the appearance of 2 kb target was retarded in PCR amplification of N213R mutant protein, indicating that 213th residue is playing a critical role in regulating the processivity and the change of the residue to aspartate could affect the processivity positively.

It was thought that the increased processivity of N213D could be accompanied by the decreased fidelity in PCR amplification despite similar exonuclease activity. To address the issue, the error rate of N213D was determined in comparison with TNA1_pol wild type protein and rTaq DNA polymerase. As shown in Table 2, the mutant showed a little less fidelity compared to wild type protein, introducing an average of one incorrect by every 3 kb (Table 2). However, the fidilty was still sixfold lower error rate than rTaq DNA polymerase (Table 2). It is not fully understood why N213D mutation increased the processivity at this point, and the structural determination may help.

### INDUSTRIAL APPLICABILITY

As described above, the present invention relates to DNA polymerases which are produced by site-specific mutagenesis from the isolated Thermococcus sp NA1. strain, their amino acid sequences, genes encoding said mutant DNA polymerases, their nucleotide sequences, preparation methods thereof and use of PCR using thereof. As mutant DNA polymerases according to the present invention have the increased processivity by site-specific mutagenesis on exonuclease active site, the present invention is broadly applicable for PCR in various molecular genetic technologies.

1. A DNA polymerase comprising any one amino acids sequence selected from the group consisting of from 3 to 9 of SEQ ID NO: 1, from 3 to 9 of SEQ ID NO: 2, from 3 to 9 of SEQ ID NO: 3, from 3 to 9 of SEQ ID NO: 4, from 3 to 9 of SEQ ID NO: 5, from 3 to 9 of SEQ ID NO: 6, from 3 to 9 of SEQ ID NO: 7, from 3 to 9 of SEQ ID NO: 8, from 3 to 9 of SEQ ID NO: 9, from 3 to 9 of SEQ ID NO: 10, from 3 to 9 of SEQ ID NO: 11 and from 3 to 9 of SEQ ID NO: 12.

2. A DNA polymerase comprising any one amino acids sequence selected from the group consisting of SEQ ID NO: 1 to SEQ ID NO: 12.

3. A DNA polymerase consisting of any one amino acids sequence selected from the group consisting of SEQ ID NO: 13 to SEQ ID NO: 24.

4. A nucleotide sequence encoding the DNA polymerase of any one amino acids sequence selected from the group consisting of SEQ ID NO: 13 to SEQ ID NO: 24.

5. A method for DNA polymerization by using the DNA polymerase of claim 1.

6. A method for PCR by using the DNA polymerase of claim 1.

7. A recombinant vector comprising the nucleotide sequence encoding the DNA polymerase of claim 4.

8. A host cell transformed with the recombinant vector of claim 7.

9. A method for producing the DNA polymerase comprising the steps: (a) culturing the host cell of claim 8; (b) inducing expression of a recombinant protein; and (c) purifying the DNA polymerase protein.

10. A method for DNA polymerization by using the DNA polymerase of claim 2.

11. A method for DNA polymerization by using the DNA polymerase of claim 3.

12. A method for DNA polymerization by using the DNA polymerase of claim 4.

13. A method for PCR by using the DNA polymerase of claim 2.

14. A method for PCR by using the DNA polymerase of claim 3.

15. A method for PCR by using the DNA polymerase of claim 4.


SEQUENCE LISTING (36 sequences)

SEQ ID NO: 1 - protein, 11 aa, Thermococcus sp. NA1
KMLAFAIETLY

SEQ ID NO: 2 - protein, 11 aa, Thermococcus sp. NA1
VLITYDGDNFD

SEQ ID NO: 3 - protein, 11 aa, Thermococcus sp. NA1
LITYNDDNFDF

SEQ ID NO: 4 - protein, 11 aa, Thermococcus sp. NA1
ITYNGANFDFA

SEQ ID NO: 5 - protein, 11 aa, Thermococcus sp. NA1
TYNGDAFDFAY

SEQ ID NO: 6 - protein, 11 aa, Thermococcus sp. NA1
TYNGDDFDFAY

SEQ ID NO: 7 - protein, 11 aa, Thermococcus sp. NA1
TYNGDEFDFAY

SEQ ID NO: 8 - protein, 11 aa, Thermococcus sp. NA1
TYNGDFFDFAY

SEQ ID NO: 9 - protein, 11 aa, Thermococcus sp. NA1
TYNGDRFDFAY

SEQ ID NO: 10 - protein, 11 aa, Thermococcus sp. NA1
YNGDNDDFAYL

SEQ ID NO: 11 - protein, 11 aa, Thermococcus sp. NA1
NGDNFAFAYLK

SEQ ID NO: 12 - protein, 11 aa, Thermococcus sp. NA1
ERVARYSMEDA

SEQ ID NO: 13 - protein, 773 aa, Thermococcus sp. NA1
MILDVDYITEDGKPVIRIFKKEKGEFKIEYDRDFEPYIYALLKDDSAIEEVKKITAERHGKVVKVKRAEKVNKKFLGRPVEVWKLYFEHPQDVPAIRDKIRAHPGVIDIYEYDIPFAKRYLIDKGLVPMEGDEELKMLAFAIETLYHEGEEFGTGPILMISYADENEARVITWKKIDLPYVDVVSTEKEMIKRFLRVVKEKDPDVLITYNGDNFDFAYLKKRCEKLGISFTLGRDGSEPKIHRMGDRFAVEVKGRIHFDLYPVIRRTINLPTYTLEVVYEAVFGKPKEKVYAEEITLAWESGEGLERVARYSMEDAKATYELGREFFPMEAQLSRLIGQSLWDVSRSSTGNLVEWFLLRKAYERNELAPNKPDEGELARRRNSYAGGYVKEPERGLWDNIVYLDFRSLYPSIIITHNVSPDTLNREGCKEYDVAPQVGHKFCKDFPGFIPSLLGNLLEERQKIKRKMKATIDPLEKKLLDYRQRAIKILANSYYGYYGYPRARWYCKECAESVTAWGREYIEMTIREIEEKYGFKVLYADTDGFYATIPGADAETVKKKAKEFLKYINAKLPGLLELEYEGFYKRGFFVTKKKYAVIDEEGKIVTRGLEIVRRDWSDIAKETQARVLEALLKDGNVEKAVKIVKEITEKLSKYEIPPEKLVIHEQITRELKDYKATGPHVAIAKRLAARGIKVRPGTIISYIVLKGSGRIGDRAIPFDEFDPTKHKYDADYYIENQVLPAVMRILEAFGYKKEDLRYQKTRQVGLGAWLKPKK

SEQ ID NO: 14 - protein, 773 aa, Thermococcus sp. NA1
MILDVDYITEDGKPVIRIFKKEKGEFKIEYDRDFEPYIYALLKDDSAIEEVKKITAERHGKVVKVKRAEKVNKKFLGRPVEVWKLYFEHPQDVPAIRDKIRAHPGVIDIYEYDIPFAKRYLIDKGLVPMEGDEELKMLAFDIETLYHEGEEFGTGPILMISYADENEARVITWKKIDLPYVDVVSTEKEMIKRFLRVVKEKDPDVLITYDGDNFDFAYLKKRCEKLGISFTLGRDGSEPKIHRMGDRFAVEVKGRIHFDLYPVIRRTINLPTYTLEVVYEAVFGKPKEKVYAEEITLAWESGEGLERVARYSMEDAKATYELGREFFPMEAQLSRLIGQSLWDVSRSSTGNLVEWFLLRKAYERNELAPNKPDEGELARRRNSYAGGYVKEPERGLWDNIVYLDFRSLYPSIIITHNVSPDTLNREGCKEYDVAPQVGHKFCKDFPGFIPSLLGNLLEERQKIKRKMKATIDPLEKKLLDYRQRAIKILANSYYGYYGYPRARWYCKECAESVTAWGREYIEMTIREIEEKYGFKVLYADTDGFYATIPGADAETVKKKAKEFLKYINAKLPGLLELEYEGFYKRGFFVTKKKYAVIDEEGKIVTRGLEIVRRDWSDIAKETQARVLEALLKDGNVEKAVKIVKEITEKLSKYEIPPEKLVIHEQITRELKDYKATGPHVAIAKRLAARGIKVRPGTIISYIVLKGSGRIGDRAIPFDEFDPTKHKYDADYYIENQVLPAVMRILEAFGYKKEDLRYQKTRQVGLGAWLKPKK

SEQ ID NO: 15 - protein, 773 aa, Thermococcus sp. NA1
MILDVDYITEDGKPVIRIFKKEKGEFKIEYDRDFEPYIYALLKDDSAIEEVKKITAERHGKVVKVKRAEKVNKKFLGRPVEVWKLYFEHPQDVPAIRDKIRAHPGVIDIYEYDIPFAKRYLIDKGLVPMEGDEELKMLAFDIETLYHEGEEFGTGPILMISYADENEARVITWKKIDLPYVDVVSTEKEMIKRFLRVVKEKDPDVLITYNDDNFDFAYLKKRCEKLGISFTLGRDGSEPKIHRMGDRFAVEVKGRIHFDLYPVIRRTINLPTYTLEVVYEAVFGKPKEKVYAEEITLAWESGEGLERVARYSMEDAKATYELGREFFPMEAQLSRLIGQSLWDVSRSSTGNLVEWFLLRKAYERNELAPNKPDEGELARRRNSYAGGYVKEPERGLWDNIVYLDFRSLYPSIIITHNVSPDTLNREGCKEYDVAPQVGHKFCKDFPGFIPSLLGNLLEERQKIKRKMKATIDPLEKKLLDYRQRAIKILANSYYGYYGYPRARWYCKECAESVTAWGREYIEMTIREIEEKYGFKVLYADTDGFYATIPGADAETVKKKAKEFLKYINAKLPGLLELEYEGFYKRGFFVTKKKYAVIDEEGKIVTRGLEIVRRDWSDIAKETQARVLEALLKDGNVEKAVKIVKEITEKLSKYEIPPEKLVIHEQITRELKDYKATGPHVAIAKRLAARGIKVRPGTIISYIVLKGSGRIGDRAIPFDEFDPTKHKYDADYYIENQVLPAVMRILEAFGYKKEDLRYQKTRQVGLGAWLKPKK

SEQ ID NO: 16 - protein, 773 aa, Thermococcus sp. NA1
MILDVDYITEDGKPVIRIFKKEKGEFKIEYDRDFEPYIYALLKDDSAIEEVKKITAERHGKVVKVKRAEKVNKKFLGRPVEVWKLYFEHPQDVPAIRDKIRAHPGVIDIYEYDIPFAKRYLIDKGLVPMEGDEELKMLAFDIETLYHEGEEFGTGPILMISYADENEARVITWKKIDLPYVDVVSTEKEMIKRFLRVVKEKDPDVLITYNGANFDFAYLKKRCEKLGISFTLGRDGSEPKIHRMGDRFAVEVKGRIHFDLYPVIRRTINLPTYTLEVVYEAVFGKPKEKVYAEEITLAWESGEGLERVARYSMEDAKATYELGREFFPMEAQLSRLIGQSLWDVSRSSTGNLVEWFLLRKAYERNELAPNKPDEGELARRRNSYAGGYVKEPERGLWDNIVYLDFRSLYPSIIITHNVSPDTLNREGCKEYDVAPQVGHKFCKDFPGFIPSLLGNLLEERQKIKRKMKATIDPLEKKLLDYRQRAIKILANSYYGYYGYPRARWYCKECAESVTAWGREYIEMTIREIEEKYGFKVLYADTDGFYATIPGADAETVKKKAKEFLKYINAKLPGLLELEYEGFYKRGFFVTKKKYAVIDEEGKIVTRGLEIVRRDWSDIAKETQARVLEALLKDGNVEKAVKIVKEITEKLSKYEIPPEKLVIHEQITRELKDYKATGPHVAIAKRLAARGIKVRPGTIISYIVLKGSGRIGDRAIPFDEFDPTKHKYDADYYIENQVLPAVMRILEAFGYKKEDLRYQKTRQVGLGAWLKPKK

SEQ ID NO: 17 - protein, 773 aa, Thermococcus sp. NA1
MILDVDYITEDGKPVIRIFKKEKGEFKIEYDRDFEPYIYALLKDDSAIEEVKKITAERHGKVVKVKRAEKVNKKFLGRPVEVWKLYFEHPQDVPAIRDKIRAHPGVIDIYEYDIPFAKRYLIDKGLVPMEGDEELKMLAFDIETLYHEGEEFGTGPILMISYADENEARVITWKKIDLPYVDVVSTEKEMIKRFLRVVKEKDPDVLITYNGDAFDFAYLKKRCEKLGISFTLGRDGSEPKIHRMGDRFAVEVKGRIHFDLYPVIRRTINLPTYTLEVVYEAVFGKPKEKVYAEEITLAWESGEGLERVARYSMEDAKATYELGREFFPMEAQLSRLIGQSLWDVSRSSTGNLVEWFLLRKAYERNELAPNKPDEGELARRRNSYAGGYVKEPERGLWDNIVYLDFRSLYPSIIITHNVSPDTLNREGCKEYDVAPQVGHKFCKDFPGFIPSLLGNLLEERQKIKRKMKATIDPLEKKLLDYRQRAIKILANSYYGYYGYPRARWYCKECAESVTAWGREYIEMTIREIEEKYGFKVLYADTDGFYATIPGADAETVKKKAKEFLKYINAKLPGLLELEYEGFYKRGFFVTKKKYAVIDEEGKIVTRGLEIVRRDWSDIAKETQARVLEALLKDGNVEKAVKIVKEITEKLSKYEIPPEKLVIHEQITRELKDYKATGPHVAIAKRLAARGIKVRPGTIISYIVLKGSGRIGDRAIPFDEFDPTKHKYDADYYIENQVLPAVMRILEAFGYKKEDLRYQKTRQVGLGAWLKPKK

SEQ ID NO: 18 - protein, 773 aa, Thermococcus sp. NA1
MILDVDYITEDGKPVIRIFKKEKGEFKIEYDRDFEPYIYALLKDDSAIEEVKKITAERHGKVVKVKRAEKVNKKFLGRPVEVWKLYFEHPQDVPAIRDKIRAHPGVIDIYEYDIPFAKRYLIDKGLVPMEGDEELKMLAFDIETLYHEGEEFGTGPILMISYADENEARVITWKKIDLPYVDVVSTEKEMIKRFLRVVKEKDPDVLITYNGDDFDFAYLKKRCEKLGISFTLGRDGSEPKIHRMGDRFAVEVKGRIHFDLYPVIRRTINLPTYTLEVVYEAVFGKPKEKVYAEEITLAWESGEGLERVARYSMEDAKATYELGREFFPMEAQLSRLIGQSLWDVSRSSTGNLVEWFLLRKAYERNELAPNKPDEGELARRRNSYAGGYVKEPERGLWDNIVYLDFRSLYPSIIITHNVSPDTLNREGCKEYDVAPQVGHKFCKDFPGFIPSLLGNLLEERQKIKRKMKATIDPLEKKLLDYRQRAIKILANSYYGYYGYPRARWYCKECAESVTAWGREYIEMTIREIEEKYGFKVLYADTDGFYATIPGADAETVKKKAKEFLKYINAKLPGLLELEYEGFYKRGFFVTKKKYAVIDEEGKIVTRGLEIVRRDWSDIAKETQARVLEALLKDGNVEKAVKIVKEITEKLSKYEIPPEKLVIHEQITRELKDYKATGPHVAIAKRLAARGIKVRPGTIISYIVLKGSGRIGDRAIPFDEFDPTKHKYDADYYIENQVLPAVMRILEAFGYKKEDLRYQKTRQVGLGAWLKPKK

SEQ ID NO: 19 - protein, 773 aa, Thermococcus sp. NA1
MILDVDYITEDGKPVIRIFKKEKGEFKIEYDRDFEPYIYALLKDDSAIEEVKKITAERHGKVVKVKRAEKVNKKFLGRPVEVWKLYFEHPQDVPAIRDKIRAHPGVIDIYEYDIPFAKRYLIDKGLVPMEGDEELKMLAFDIETLYHEGEEFGTGPILMISYADENEARVITWKKIDLPYVDVVSTEKEMIKRFLRVVKEKDPDVLITYNGDEFDFAYLKKRCEKLGISFTLGRDGSEPKIHRMGDRFAVEVKGRIHFDLYPVIRRTINLPTYTLEVVYEAVFGKPKEKVYAEEITLAWESGEGLERVARYSMEDAKATYELGREFFPMEAQLSRLIGQSLWDVSRSSTGNLVEWFLLRKAYERNELAPNKPDEGELARRRNSYAGGYVKEPERGLWDNIVYLDFRSLYPSIIITHNVSPDTLNREGCKEYDVAPQVGHKFCKDFPGFIPSLLGNLLEERQKIKRKMKATIDPLEKKLLDYRQRAIKILANSYYGYYGYPRARWYCKECAESVTAWGREYIEMTIREIEEKYGFKVLYADTDGFYATIPGADAETVKKKAKEFLKYINAKLPGLLELEYEGFYKRGFFVTKKKYAVIDEEGKIVTRGLEIVRRDWSDIAKETQARVLEALLKDGNVEKAVKIVKEITEKLSKYEIPPEKLVIHEQITRELKDYKATGPHVAIAKRLAARGIKVRPGTIISYIVLKGSGRIGDRAIPFDEFDPTKHKYDADYYIENQVLPAVMRILEAFGYKKEDLRYQKTRQVGLGAWLKPKK

SEQ ID NO: 20 - protein, 773 aa, Thermococcus sp. NA1
MILDVDYITEDGKPVIRIFKKEKGEFKIEYDRDFEPYIYALLKDDSAIEEVKKITAERHGKVVKVKRAEKVNKKFLGRPVEVWKLYFEHPQDVPAIRDKIRAHPGVIDIYEYDIPFAKRYLIDKGLVPMEGDEELKMLAFDIETLYHEGEEFGTGPILMISYADENEARVITWKKIDLPYVDVVSTEKEMIKRFLRVVKEKDPDVLITYNGDFFDFAYLKKRCEKLGISFTLGRDGSEPKIHRMGDRFAVEVKGRIHFDLYPVIRRTINLPTYTLEVVYEAVFGKPKEKVYAEEITLAWESGEGLERVARYSMEDAKATYELGREFFPMEAQLSRLIGQSLWDVSRSSTGNLVEWFLLRKAYERNELAPNKPDEGELARRRNSYAGGYVKEPERGLWDNIVYLDFRSLYPSIIITHNVSPDTLNREGCKEYDVAPQVGHKFCKDFPGFIPSLLGNLLEERQKIKRKMKATIDPLEKKLLDYRQRAIKILANSYYGYYGYPRARWYCKECAESVTAWGREYIEMTIREIEEKYGFKVLYADTDGFYATIPGADAETVKKKAKEFLKYINAKLPGLLELEYEGFYKRGFFVTKKKYAVIDEEGKIVTRGLEIVRRDWSDIAKETQARVLEALLKDGNVEKAVKIVKEITEKLSKYEIPPEKLVIHEQITRELKDYKATGPHVAIAKRLAARGIKVRPGTIISYIVLKGSGRIGDRAIPFDEFDPTKHKYDADYYIENQVLPAVMRILEAFGYKKEDLRYQKTRQVGLGAWLKPKK

SEQ ID NO: 21 - protein, 773 aa, Thermococcus sp. NA1
MILDVDYITEDGKPVIRIFKKEKGEFKIEYDRDFEPYIYALLKDDSAIEEVKKITAERHGKVVKVKRAEKVNKKFLGRPVEVWKLYFEHPQDVPAIRDKIRAHPGVIDIYEYDIPFAKRYLIDKGLVPMEGDEELKMLAFDIETLYHEGEEFGTGPILMISYADENEARVITWKKIDLPYVDVVSTEKEMIKRFLRVVKEKDPDVLITYNGDRFDFAYLKKRCEKLGISFTLGRDGSEPKIHRMGDRFAVEVKGRIHFDLYPVIRRTINLPTYTLEVVYEAVFGKPKEKVYAEEITLAWESGEGLERVARYSMEDAKATYELGREFFPMEAQLSRLIGQSLWDVSRSSTGNLVEWFLLRKAYERNELAPNKPDEGELARRRNSYAGGYVKEPERGLWDNIVYLDFRSLYPSIIITHNVSPDTLNREGCKEYDVAPQVGHKFCKDFPGFIPSLLGNLLEERQKIKRKMKATIDPLEKKLLDYRQRAIKILANSYYGYYGYPRARWYCKECAESVTAWGREYIEMTIREIEEKYGFKVLYADTDGFYATIPGADAETVKKKAKEFLKYINAKLPGLLELEYEGFYKRGFFVTKKKYAVIDEEGKIVTRGLEIVRRDWSDIAKETQARVLEALLKDGNVEKAVKIVKEITEKLSKYEIPPEKLVIHEQITRELKDYKATGPHVAIAKRLAARGIKVRPGTIISYIVLKGSGRIGDRAIPFDEFDPTKHKYDADYYIENQVLPAVMRILEAFGYKKEDLRYQKTRQVGLGAWLKPKK

SEQ ID NO: 22 - protein, 773 aa, Thermococcus sp. NA1
MILDVDYITEDGKPVIRIFKKEKGEFKIEYDRDFEPYIYALLKDDSAIEEVKKITAERHGKVVKVKRAEKVNKKFLGRPVEVWKLYFEHPQDVPAIRDKIRAHPGVIDIYEYDIPFAKRYLIDKGLVPMEGDEELKMLAFDIETLYHEGEEFGTGPILMISYADENEARVITWKKIDLPYVDVVSTEKEMIKRFLRVVKEKDPDVLITYNGDNDDFAYLKKRCEKLGISFTLGRDGSEPKIHRMGDRFAVEVKGRIHFDLYPVIRRTINLPTYTLEVVYEAVFGKPKEKVYAEEITLAWESGEGLERVARYSMEDAKATYELGREFFPMEAQLSRLIGQSLWDVSRSSTGNLVEWFLLRKAYERNELAPNKPDEGELARRRNSYAGGYVKEPERGLWDNIVYLDFRSLYPSIIITHNVSPDTLNREGCKEYDVAPQVGHKFCKDFPGFIPSLLGNLLEERQKIKRKMKATIDPLEKKLLDYRQRAIKILANSYYGYYGYPRARWYCKECAESVTAWGREYIEMTIREIEEKYGFKVLYADTDGFYATIPGADAETVKKKAKEFLKYINAKLPGLLELEYEGFYKRGFFVTKKKYAVIDEEGKIVTRGLEIVRRDWSDIAKETQARVLEALLKDGNVEKAVKIVKEITEKLSKYEIPPEKLVIHEQITRELKDYKATGPHVAIAKRLAARGIKVRPGTIISYIVLKGSGRIGDRAIPFDEFDPTKHKYDADYYIENQVLPAVMRILEAFGYKKEDLRYQKTRQVGLGAWLKPKK

SEQ ID NO: 23 - protein, 773 aa, Thermococcus sp. NA1
MILDVDYITEDGKPVIRIFKKEKGEFKIEYDRDFEPYIYALLKDDSAIEEVKKITAERHGKVVKVKRAEKVNKKFLGRPVEVWKLYFEHPQDVPAIRDKIRAHPGVIDIYEYDIPFAKRYLIDKGLVPMEGDEELKMLAFDIETLYHEGEEFGTGPILMISYADENEARVITWKKIDLPYVDVVSTEKEMIKRFLRVVKEKDPDVLITYNGDNFAFAYLKKRCEKLGISFTLGRDGSEPKIHRMGDRFAVEVKGRIHFDLYPVIRRTINLPTYTLEVVYEAVFGKPKEKVYAEEITLAWESGEGLERVARYSMEDAKATYELGREFFPMEAQLSRLIGQSLWDVSRSSTGNLVEWFLLRKAYERNELAPNKPDEGELARRRNSYAGGYVKEPERGLWDNIVYLDFRSLYPSIIITHNVSPDTLNREGCKEYDVAPQVGHKFCKDFPGFIPSLLGNLLEERQKIKRKMKATIDPLEKKLLDYRQRAIKILANSYYGYYGYPRARWYCKECAESVTAWGREYIEMTIREIEEKYGFKVLYADTDGFYATIPGADAETVKKKAKEFLKYINAKLPGLLELEYEGFYKRGFFVTKKKYAVIDEEGKIVTRGLEIVRRDWSDIAKETQARVLEALLKDGNVEKAVKIVKEITEKLSKYEIPPEKLVIHEQITRELKDYKATGPHVAIAKRLAARGIKVRPGTIISYIVLKGSGRIGDRAIPFDEFDPTKHKYDADYYIENQVLPAVMRILEAFGYKKEDLRYQKTRQVGLGAWLKPKK

SEQ ID NO: 24 - protein, 773 aa, Thermococcus sp. NA1
MILDVDYITEDGKPVIRIFKKEKGEFKIEYDRDFEPYIYALLKDDSAIEEVKKITAERHGKVVKVKRAEKVNKKFLGRPVEVWKLYFEHPQDVPAIRDKIRAHPGVIDIYEYDIPFAKRYLIDKGLVPMEGDEELKMLAFDIETLYHEGEEFGTGPILMISYADENEARVITWKKIDLPYVDVVSTEKEMIKRFLRVVKEKDPDVLITYNGDNFDFAYLKKRCEKLGISFTLGRDGSEPKIHRMGDRFAVEVKGRIHFDLYPVIRRTINLPTYTLEVVYEAVFGKPKEKVYAEEITLAWESGEGLERVARFSMEDAKATYELGREFFPMEAQLSRLIGQSLWDVSRSSTGNLVEWFLLRKAYERNELAPNKPDEGELARRRNSYAGGYVKEPERGLWDNIVYLDFRSLYPSIIITHNVSPDTLNREGCKEYDVAPQVGHKFCKDFPGFIPSLLGNLLEERQKIKRKMKATIDPLEKKLLDYRQRAIKILANSYYGYYGYPRARWYCKECAESVTAWGREYIEMTIREIEEKYGFKVLYADTDGFYATIPGADAETVKKKAKEFLKYINAKLPGLLELEYEGFYKRGFFVTKKKYAVIDEEGKIVTRGLEIVRRDWSDIAKETQARVLEALLKDGNVEKAVKIVKEITEKLSKYEIPPEKLVIHEQITRELKDYKATGPHVAIAKRLAARGIKVRPGTIISYIVLKGSGRIGDRAIPFDEFDPTKHKYDADYYIENQVLPAVMRILEAFGYKKEDLRYQKTRQVGLGAWLKPKK

SEQ ID NO: 25 - DNA, 39 bp, Artificial Sequence
  primer
atgctcgcctttgccatcgagacgctctaccacgagggc

SEQ ID NO: 26 - DNA, 39 bp, Artificial Sequence
  primer
ctcattacctacgacggcgacaactttgactttgcttac

SEQ ID NO: 27 - DNA, 39 bp, Artificial Sequence
  primer
attacctacaacgacgacaactttgactttgcttacctc

SEQ ID NO: 28 - DNA, 36 bp, Artificial Sequence
  primer
acctacaacggcgccaactttgactttgcttacctc

SEQ ID NO: 29 - DNA, 33 bp, Artificial Sequence
  primer
tacaacggcgacgcctttgactttgcttacctc

SEQ ID NO: 30 - DNA, 33 bp, Artificial Sequence
  primer
tacaacggcgacgactttgactttgcttacctc

SEQ ID NO: 31 - DNA, 33 bp, Artificial Sequence
  primer
tacaacggcgacgagtttgactttgcttacctc

SEQ ID NO: 32 - DNA, 33 bp, Artificial Sequence
  primer
tacaacggcgacttctttgactttgcttacctc

SEQ ID NO: 33 - DNA, 33 bp, Artificial Sequence
  primer
tacaacggcgaccgctttgactttgcttacctc

SEQ ID NO: 34 - DNA, 38 bp, Artificial Sequence
  primer
aacggcgacaacgacgactttgcttacctcaaaaaacg

SEQ ID NO: 35 - DNA, 39 bp, Artificial Sequence
  primer
ggcgacaactttgcctttgcttacctcaaaaaacgttgc

SEQ ID NO: 36 - DNA, 39 bp, Artificial Sequence
  primer
cgcgttgcgcgcttctctatggaagatgcaaaggcaacc